(19) United States
(12) Patent Application Publication
(10) Pub. No.: US 2007/0082052 A1
(43) Pub. Date: Apr. 12, 2007

(54) Tympanic membrane repair constructs

(76) Inventors: Lawrence J. Bonassar, Ithaca, NY (US); Cliff Megerian, Shaker Heights, OH (US); Richard M. Beane, Hingham, MA (US)

Correspondence Address: FISH & RICHARDSON PC, P.O. BOX 1022, MINNEAPOLIS, MN, 55440-1022, US

(21) Appl. No.: 11/475,324

(22) Filed: Jun. 26, 2006

Related U.S. Application Data
(63) Continuation-in-part of application No. 11/239,860, filed on Sep. 29, 2005.
Application No. 11/239,860 is a continuation of application No. 10/081,360, filed on Feb. 21, 2002, now abandoned.
(60) Provisional application No. 60/271,105, filed on Feb. 23, 2001.

(51) Int. Cl. A61K 9/14

(52) U.S. Cl. 424/486; 424/488

(57) ABSTRACT

The invention features methods of making hydrogel constructs that can be used to repair perforations in tympanic membranes, the hydrogel constructs themselves, and methods of repair.

### CROSS-REFERENCE TO RELATED APPLICATIONS

This application is a continuation-in-part of U.S. patent application Ser. No. 11/239,860, filed on Sep. 29, 2005, now pending, which is a continuation of U.S. patent application Ser. No. 10/081,360, filed on Feb. 21, 2002, now abandoned, and claims the benefit of U.S. Provisional Patent Application Ser. No. 60/271,105, filed on Feb. 23, 2001, the contents of all of which are incorporated herein by reference in their entireties.

### TECHNICAL FIELD

This invention relates to degradable hydrogel constructs for use as tympanic membrane repair constructs, and methods for use thereof.

### BACKGROUND

The treatment of recurrent otitis media (middle ear infection) in children often requires placement of tubes in the tympanic membrane that facilitate drainage of fluid from the ear. Removal of these tubes after treatment results in holes in the tympanic membrane that do not heal in a significant fraction of cases (10-20%). Overall, the number of patients who require tympanostomy tubes estimated at 2,000,000 patients per year (Isaacson and Rosenfeld, Ped. Otolaryngol., 43:1183, 1996). Of these, it is estimated that 3.5-10% (70,000-200,000 patients/year) will develop persistent tympanic perforations requiring patch treatments (Golz et al., Otolaryngol., 120:524, 1999).

Standard procedures for filling such perforations involves placing a small piece of paper or other synthetic patch over the perforation, generally secured with sutures or glue, with uncertain efficacy. A more invasive procedure requires sculpting auricular cartilage or temporalis muscular fascia harvested from the patient to fit into the tympanic membrane defect. This sculpting procedure is time consuming, inexact, and difficult to reproduce and requires time in an operating room suite.

Tissue engineering involves the regeneration of tissues such as bone and cartilage by seeding cells onto a customized biodegradable polymer scaffold to provide a three dimensional environment that promotes matrix production. This structure anchors cells and permits nutrition and gas exchange with the ultimate formation of new tissue in the shape of the polymer material. See, e.g., Vacanti et al., 1994, Transplant. Proc., 26:3309-3310; and Puelacher et al., 1994, Biomaterials, 15:774-778.

### SUMMARY

The invention is based on the discovery that industrial design and manufacturing techniques, such as injection molding, can be used to create detailed, three-dimensional degradable hydrogel constructs for repairing holes in the tympanic membrane, colloquially known as the eardrum. In various embodiments, the constructs are made of a degradable hydrogel, either without cells or with cells, e.g., chondrocytes and fibroblasts. In some embodiments, the new methods involve the use of tissue engineering technology to generate precisely shaped implants or constructs to fill the perforations, using scaffold molding and cell/polymer injection molding techniques.

In one aspect, the invention features hydrogel constructs or implants for repairing a perforation in a tympanic membrane, wherein the hydrogel constructs have a defined, e.g., predetermined, shape suitable for repairing a perforation in a tympanic membrane, i.e., a biflanged construct of the general shape illustrated in FIGS. 2-3. Though the cross-section of the exemplary structure is circular, other shapes can also be used, e.g., square, rectangular, or irregular, to optimize the fit of the construct in the membrane perforation.

The degradable hydrogel constructs include a solidified hydrogel. The hydrogel can be or include, e.g., polysaccharides, proteins, polyphosphazenes, poly(oxy-ethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers. In some embodiments, the hydrogel is alginate, chitosan, pluronic, collagen, or agarose. If the hydrogel is alginate, the concentration can be from 0.5% to 8%, e.g., from 1% to 4%, e.g., approximately 2%.

In another aspect, the invention features methods of making a construct for repairing a perforation in a tympanic membrane by providing a negative mold having a defined, e.g., predetermined, negative shape of the construct, wherein the construct has the general shape illustrated in FIG. 2.

In some embodiments, the methods include introducing a liquid hydrogel composition into the mold; inducing gel formation to solidify the liquid hydrogel composition to form a hydrogel construct; and removing the hydrogel construct from the mold after gel formation, wherein the construct has a shape suitable for repairing a perforation in a tympanic membrane.

In some embodiments, the methods include suspending isolated tissue precursor cells in the liquid hydrogel to form a liquid hydrogel-precursor cell composition; introducing the liquid hydrogel-precursor cell composition into the mold; inducing gel formation to solidify the liquid hydrogel-precursor cell composition to form a construct comprising living cells, i.e., a living tissue construct; and removing the construct from the mold after gel formation, wherein the construct has a shape suitable for repairing a perforation in a tympanic membrane.

In these methods, the tissue precursor cells can be chondrocytes or fibroblasts, or a combination thereof, and the hydrogel can be alginate, chitosan, pluronic, collagen, or agarose.

Gel formation can be induced where necessary by contacting the liquid hydrogel with a suitable concentration of a divalent cation, such as Ca⁺⁺, e.g., at a concentration of about 0.2 mg/ml of alginate solution. In embodiments including tissue precursor cells, the construct can be cultured in the solidified hydrogel construct, e.g., in vitro, for a period of 1 to 30 days prior to implantation. In these methods, the negative mold can be prepared using CAD/CAM or rapid prototyping.

In a further aspect, the invention features methods for repairing a perforation in a tympanic membrane in a mammal by providing a suitable negative mold having a negative shape of the hydrogel construct; introducing a liquid hydrogel composition into the mold; inducing gel formation to solidify the liquid hydrogel composition to form a hydrogel construct; removing the hydrogel construct from the mold after gel formation; and implanting the construct into the perforation in the tympanic membrane in the mammal.

In some embodiments, the methods include suspending isolated tissue precursor cells in a hydrogel to form a liquid hydrogel-precursor cell composition; introducing the liquid hydrogel-precursor cell composition into the mold; inducing gel formation to solidify the liquid hydrogel-precursor cell composition to form a living tissue construct; removing the tissue construct from the mold after gel formation; and implanting the tissue construct into the perforation in the tympanic membrane in the mammal.

An alternative method of repairing a perforation in a tympanic membrane in a mammal includes obtaining a construct as described herein shaped to fit into the perforation; and implanting the construct into the perforation in the tympanic membrane in the mammal. In this method, the construct can be prepared by a method described herein.

The invention also features an injection-molded construct made by the methods described herein. In these methods and constructs, the hydrogels can be polysaccharides, proteins, polyphosphazenes, poly(oxy-ethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the cells evenly suspended within a mold until the gel solidifies. The hydrogels are also biocompatible, e.g., not toxic, to cells, e.g., cells suspended in the hydrogel, or in the surrounding membrane.

A “hydrogel-cell composition” is a suspension of a hydrogel containing desired tissue precursor cells. These cells can be isolated directly from a tissue source or can be obtained from a cell culture. A “tissue” is a collection or aggregation of particular cells embedded within its natural matrix, wherein the natural matrix is produced by the particular living cells. A “living tissue construct” is a collection of living cells that have a defined shape and structure. To be “living,” the cells must at least have a capacity for metabolism, but need not be able to grow or reproduce in all embodiments. Of course, a living tissue construct can also include, and in some embodiments preferably includes, cells that grow and/or reproduce. “Tissue precursor cells” are cells that form the basis of new tissue. Tissue cells can be “organ cells,” which include hepatocytes, islet cells, cells of intestinal origin, muscle cells, heart cells, cartilage cells, bone cells, kidney cells, cells of hair follicles, cells from the vitreous humor in the eyes, cells from the brain, and other cells acting primarily to synthesize and secret, or to metabolize materials. In some embodiments, these cells can be fully mature and differentiated cells. In addition, tissue precursor cells can be so-called “stem” cells or “progenitor” cells that are partially differentiated or undifferentiated precursor cells that can form a number of different types of specific cells under different ambient conditions, and that multiply and/or differentiate to form a new tissue.

An “isolated” tissue precursor cell, such as an isolated nerve cell, or an isolated nerve stem or progenitor cell or bone cell, or bone stem or progenitor cell, is a cell that has been removed from its natural environment in a tissue within an animal, and cultured in vitro, at least temporarily. The term covers single isolated cells, as well as cultures of “isolated” stem cells, that have been significantly enriched for the stem or progenitor cells with few or no differentiated cells.

As used herein, “negative mold” means a concave mold into which a liquid can be introduced for subsequent solidification. The mold is “negative” in the sense that concavity of the mold corresponds to convexity in the object to be formed.

The invention has many advantages. For example, the new methods reduce the number of manufacturing steps needed to prepare precise, three-dimensional eardrum repair constructs. In some embodiments, the methods and constructs described herein do not use living tissue or cells, in which case the manufacture and storage of the constructs is greatly simplified, and the need to obtain cells from the recipient, or HLA-matched cells, is eliminated. In some embodiments, the methods and constructs described herein do use living tissue or cells, and thereby provide increased uniformity of cell seeding throughout the construct, and increased efficiency of cell containment within the construct.

Additional advantages include: 1) elimination of variability in repair construct (“plug”) geometry due to surgical skill; 2) decrease in interoperative time by elimination of harvest and sculpting steps; 3) availability of an off-the-shelf component, which will allow for choice of variously sized implants during surgery; and 4) the fabrication of custom-designed implants via injection-molding technology.

The new technology also has significant advantages over synthetic prosthesis which have previously been used to fill these defects. Since these prior art patches must remain in place permanently for long-term efficacy, synthetic implants are less desirable due to the possibility of chronic inflammation from foreign body response. Placing the constructs described herein, rather than synthetic patches, into the defect decreases the likelihood of an immune response.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. All publications, patent applications, patents, and other references mentioned herein are incorporated by reference in their entirety. In case of conflicting subject matter, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a schematic diagram of one embodiment of the injection molding process. Immediately before injection into the mold, sterilized CaSO4 (0.2 gm/ml of alginate) in PBS was mixed with a solution of 2% alginate to initiate gel formation. The alginate/CaSO₄ mixture was injected to the sterilized mold using a syringe and needle. Formed shapes were removed from molds 15 minutes after injection.

FIG. 1B is a schematic diagram of another embodiment of the injection molding process. Bovine articular cartilage was digested in collagenase II (3 mg/ml) at 37° C. for 12-18 hours. Chondrocytes were concentrated to 1, 2.5, and 5×10⁷ cells/ml and suspended in a solution of 2% alginate. Immediately before injection into the mold, sterilized CaSO₄ (0.2 gm/ml of alginate) in PBS was mixed with chondrocytes in alginate to initiate gel formation. The chondrocyte/alginate/CaSO₄ mixture was injected to the sterilized mold using a syringe and needle. Formed shapes were removed from molds 15 minutes after injection.

FIG. 2 is an exemplary virtual template, generated by computer-aided design (CAD), of a tympanic membrane repair construct positive model that is used to prepare a negative mold. Such a model can be a computer image, or a three-dimensional, physical model.

FIG. 3 shows the fabricated calcium alginate constructs in four different sizes (1-4 mm diameter of middle portion).

FIG. 4 is a schematic illustration of a construct (2) described herein inserted in a TM (1).

FIG. 5 is a bar graph showing the percentage of healing in each treatment arm. The control group is used as an indicator for the reliability of the animal model to maintain stable TM perforation. The difference between the paper patch group and control did not reach statistical significance, whereas the improvement obtained with the 2% calcium alginate grafts was statistically significant compared to the paper patch group, and both 2% and 4% calcium alginate graft results were statistically significant as compared to the control group.

### DETAILED DESCRIPTION

The invention utilizes injection molding, and, in some embodiments, tissue-engineering techniques to generate new constructs that are used to repair holes in tympanic membranes. In contrast to conventional tissue engineering techniques that involve creating a hand-shaped scaffold and then seeding the scaffold with cells in a separate step, in some embodiments the invention utilizes a suspension of cells in a solution from which a hydrogel is formed at a controlled gelation rate. In general, negative molds of constructs used to fill perforations in the tympanic membrane are produced either by starting with a positive mold or a custom-designed drawing via computer aided design (CAD) (FIG. 2). Thereafter, standard molding materials and software are used to make negative molds from three-dimensional images or positive models. The new methods enable the formation of a variety of negative molds to vary the size and shape of the construct to fit a given patient.

The new methods can be used to grow new eardrum tissue by using a hydrogel composition that is formed into a precise shape using injection molding techniques. To guide the development and shape of the construct, a negative mold is created, and the hydrogel composition is delivered into the mold and cured to form a solid, three-dimensional construct, which is removed from the mold after the hydrogel composition is solidified, and eventually implanted into a hole in a patient's eardrum. In some embodiments, the hydrogel composition includes living cells, and in those embodiments, the construct can be first placed into an in vitro controlled environment to allow the cells to grow for a period of days or weeks within the solidified hydrogel, or the construct can be implanted directly after solidification. In the following subsections, suitable molding techniques, hydrogels, cells, and delivery methods will be described, along with illustrative examples.

In some embodiments, a precise negative mold is created to custom-make a construct of the precise size and shape for a given perforation. Alternatively, given the ability of some hydrogels to conform to a given perforation, the new constructs can also be prepared in advance in one or more set shapes, but in various sizes. A surgeon can then simply select a construct of the appropriate size and shape for a given perforation. In some embodiments, these constructs can have a cylindrical middle portion and circular flanges or discs at either end.

General Methodology

Injection molding is a common technique used for the mass production of plastic articles having complex geometry. This technique has been applied to the production of medical implants made from biocompatible materials (Konig et al., 1997, J. Biomed. Mater. Res., 38:115-119; Kapur et al., 1996, J. Biomed. Mater. Res., 33:205-216; Huggett et al., 1992, J. Prosthet. Dent. 68:634-640). Injection molding techniques have been combined with magnetic resonance imaging (MRI) and computerized tomography (CT) imaging to make custom implants based on individual anatomy (Abbott et al., 1998, Aust. Dent. J., 43:373-378; Verstreken et al., IEEE Trans. Med. Imaging. 17:842-852; Ainbinder et al., 1998, Radiol. Clin. North Am., 36:1133-1147). The application of computerized imaging techniques for manufacturing custom implants has focused on non-biological materials, including metals, ceramics and synthetic polymers.

As with any process based on injection molding, the size and shape of the construct is determined by the size and shape of the negative mold. Thus, the invention can be employed to produce a construct having essentially any size and shape, with the size and shape being precisely controlled. The size and shape of the construct can be optimized for the repair of perforations in the tympanic membrane.

Because injection molding allows for the use of a precise negative mold, detailed three-dimensional structural information from computer-aided drafting (CAD) can be used together with computer-aided manufacturing (CAM) and rapid prototyping to produce high quality molds in which the constructs are formed. CAD/CAM hardware and software are commercially available and can be employed using techniques known in the art to design and produce molds suitable for use in the invention.

Although CAD/CAM techniques can be used in the design and production of molds they are not required. In some embodiments of the invention, a mold is constructed manually, e.g., by using a Silastic ERTV mold making kit (Dow Corning). For example, negative molds can be fabricated by immersing half of a positive model in a bed formed from the mixed components of an ERTV kit. This mixture is then placed in an 80° F. oven for 30 minutes. After the bottom is hardened, approximately the same amount of uncured silastic is poured on top to a height of 2 cm. This is again cured at 80° F. for 30 minutes. After separation of the top and lower sets of the mold, the model is removed.

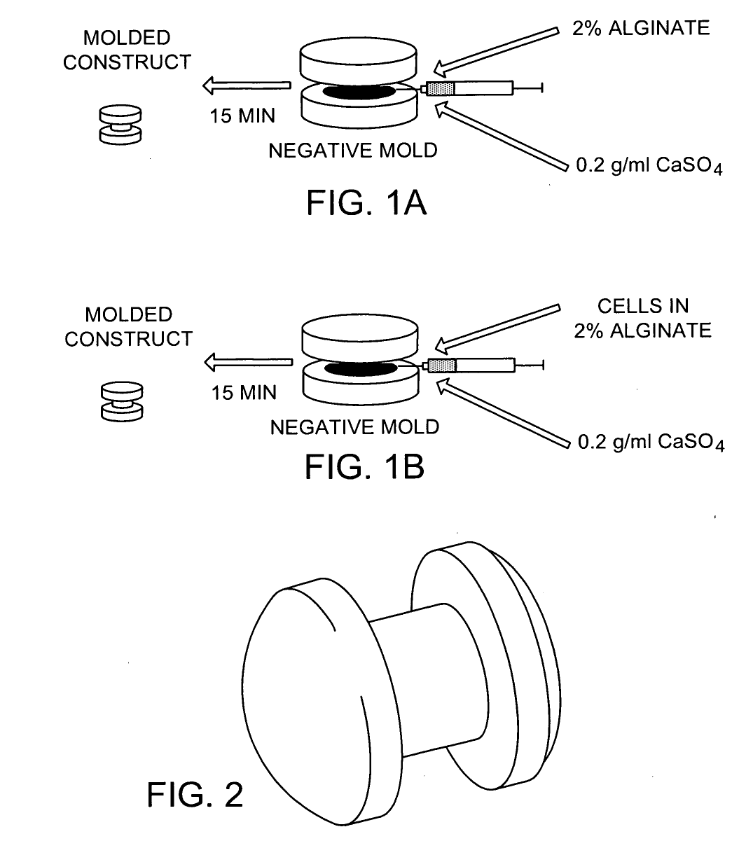

In general, the degradable hydrogel constructs described herein are unitary constructs having a first, middle portion (e.g., a shaft) having a first end and a second end. The first end is coaxially connected to a second portion having a larger diameter than the first, middle portion. The second end is coaxially connected to a third portion having a larger diameter than the first, middle portion. In some embodiments, the terminal ends of the second and third portions that are not connected to the first, middle portion are dome shaped, e.g., as shown in FIG. 2. The dome shaped ends, for example, can ease insertion into the perforation. In general, the diameter of the first, middle portion will be selected fit snugly inside the perforation that is desired to be repaired, and the length of the first, middle portion is selected such that the inner faces of the second and third portions fit snugly against the tympanic membrane. The diameter of the second and third portions is selected to allow for insertion of the construct in the hole in the membrane, and retention of the construct therein without the need for any glue or sutures; an example is illustrated in FIG. 4. In some embodiments, the general shape of the degradable hydrogel construct is a biflanged cylinder as shown in FIGS. 1-3. In some embodiments, the shape of the constructs described herein is similar to that of a standard rubber grommet, without a hole in the middle.

In some embodiments, e.g., as shown in FIG. 1B, cells are extracted from a source, such as cartilage, using standard techniques. For example, cartilage can be cut into small pieces of 1 to 3 mm³, and then disrupted with an enzyme or other chemical that separates the cells but does not destroy them. For example, collagenase works well for disrupting collagen into separate cells. Fibroblasts can be isolated from skin by a similar method. For example, the dermis can be separated from the skin and minced, and then treated with collagenase to disrupt the dermis into separate cells, which are mostly fibroblasts. In both cases, the cells are filtered to remove extracellular matrix debris, and are centrifuged and resuspended.

A combination of fibroblasts and chondrocytes is then suspended in a hydrogel, such as a diluted alginate solution (e.g., 0.1-8%), to produce a hydrogel-cell composition that can be delivered into the mold in liquid form, and is then injection molded into a pre-constructed negative mold.

When necessary, the hydrogel composition is introduced into the mold simultaneously with a precise curing composition, such as 0.2 g/ml CaSO₄. After a predetermined time, such as 15 minutes for alginate, the hydrogel composition is removed from the mold after it has solidified or cured.

The molded hydrogel construct can then be implanted directly into the patient's eardrum. Alternatively, constructs including living cells can be cultured in vitro for a time sufficient for tissue to develop. Constructs without living cells can be packaged and stored for a time until needed.

Hydrogels

Any suitable polymer hydrogel can be used in methods of the invention. A suitable polymer hydrogel is one that is biologically compatible, non-cytotoxic, and formed through controllable crosslinking (gelation), under conditions compatible with viability of any isolated cells suspended in the solution that undergoes gelation. Various polymer hydrogels meeting these requirements are known in the art and can be used in the practice of the invention. Examples of different hydrogels suitable for practicing this invention, include, but are not limited to: (1) hydrogels cross-linked by ions, e.g., sodium alginate; (2) temperature dependent hydrogels that solidify or set at body temperature, e.g., PLURONICS™; (3) hydrogels set by exposure to either visible or ultraviolet light, e.g., polyethylene glycol polylactic acid copolymers with acrylate end groups; and (4) hydrogels that are set or solidified upon a change in pH, e.g., TETRONICS™.

Examples of materials that can be used to form these different hydrogels include polysaccharides such as alginate, polyphosphazenes, and polyacrylates, which are cross-linked ionically, or block copolymers such as PLURONICS™ (also known as POLOXAMERS™), which are poly(oxyethylene)-poly(oxypropylene) block polymers solidified by changes in temperature, or TETRONICS™ (also known as POLOXAMINES™), which are poly(oxyethylene)-poly(oxypropylene) block polymers of ethylene diamine solidified by changes in pH.

Ionic Hydrogels

Ionic polysaccharides, such as alginates and chitosan, can be used in the methods described herein; these degradable hydrogels solidify when the proper concentrations of ions are added. For example, alginate is an anionic polysaccharide capable of reversible gelation in the presence of an effective concentration of a divalent cation. A hydrogel can be produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix.

In a more specific example, Ca⁺⁺ can be supplied conveniently in the form of CaSO₄. In some embodiments of the invention, CaSO₄ is added in the amount of 0.1 to 0.5 gram, e.g., approximately 0.2 gram, per milliliter of a 2% solution of alginate. If the amount of soluble alginate is increased or decreased, the amount of divalent cation may need to be adjusted accordingly. Such adjustment is within the ordinary skill in the art. The solubility of CaSO₄ is 0.209 g/ml, which is much lower than that of CaCl₂ (74.5 g/ml), which is the crosslinking agent typically used for encapsulation of cells in alginate. See Beekman et al., 1997, Exper. Cell Res., 237:135-141. At a concentration of CaSO₄ near or above the solubility limit, Ca²⁺ in solution begins to crosslink alginate, and it is replenished by solubilization of precipitated CaSO₄. This results in a significant slowing of the crosslinking process. Such slowing can be advantageous, because it allows the alginate/CaSO₄ mixture to be injected into a mold before the completion of the crosslinking process occurs in the shaped implant.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups.

Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Bioerodible or biodegradable polymers, i.e., polymers that dissolve or degrade within a period that is acceptable in the desired application (usually in vivo therapy), will degrade in less than about five years and most preferably in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described above are known to those skilled in the art. See, for example Concise Encyclopedia of Polymer Science and Engineering, J. I. Kroschwitz, editor (John Wiley and Sons, New York, N.Y., 1990). Many polymers, such as poly(acrylic acid), alginates, and PLURONICS™, are commercially available.

Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels, as described with respect to cations.

For purposes of preventing the passage of antibodies into the hydrogel, but allowing the entry of nutrients, a useful polymer size in the hydrogel is in the range of between 10,000 D and 18,500 D. Smaller polymers result in gels of higher density with smaller pores.

Temperature-Dependent Hydrogels

Temperature-dependent, or thermosensitive, hydrogels can be use in the methods of the invention. These hydrogels have so-called “reverse gelation” properties, i.e., they are liquids at or below room temperature, and gel when warmed to higher temperatures, e.g., at or above body temperature. Thus, these hydrogels can be easily injected into a mold at or below room temperature as a liquid and automatically form a semi-solid gel when warmed to or above body temperature. Examples of such temperature-dependent hydrogels are PLURONICS™ (BASF-Wyandotte), such as polyoxyethylene-polyoxypropylene F-108, F-68, and F-127, poly (N-isopropylacrylamide), and N-isopropylacrylamide copolymers.

These copolymers can be manipulated by standard techniques to affect their physical properties such as porosity, rate of degradation, transition temperature, and degree of rigidity. For example, the addition of low molecular weight saccharides in the presence and absence of salts affects the lower critical solution temperature (LCST) of typical thermosensitive polymers. In addition, when these gels are prepared at concentrations ranging between 5 and 25% (W/V) by dispersion at 4° C., the viscosity and the gel-sol transition temperature are affected, the gel-sol transition temperature being inversely related to the concentration. These gels have diffusion characteristics capable of allowing cells to survive and be nourished.

U.S. Pat. No. 4,188,373 describes using PLURONIC™ polyols in aqueous compositions to provide thermal gelling aqueous systems. U.S. Pat. Nos. 4,474,751, '752, '753, and 4,478,822 describe drug delivery systems which utilize thermosetting polyoxyalkylene gels; with these systems, both the gel transition temperature and/or the rigidity of the gel can be modified by adjustment of the pH and/or the ionic strength, as well as by the concentration of the polymer.

pH-Dependent Hydrogels

Other hydrogels suitable for use in the methods of the invention are pH-dependent. These hydrogels are liquids at, below, or above specific pH values, and gel when exposed to specific pHs, e.g., 7.35 to 7.45, the normal pH range of extracellular fluids within the human body. Thus, these hydrogels can be easily delivered into a mold as a liquid and form a semi-solid gel when exposed to the proper pH. Examples of such pH-dependent hydrogels are TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate). These copolymers can be manipulated by standard techniques to affect their physical properties.

An example of another a useful pH-dependent hydrogel is collagen. Collagen is a protein that undergoes cross-linking in response to shift in pH from alkaline to acid, e.g., a shift from a pH in the range of <2 to a pH in the range of >6. See, e.g., Bell et al., 1979, Proc. Nat. Acad. Sci., 76:1274.

Light Solidified Hydrogels

Other hydrogels that can be used in the methods of the invention are solidified by either visible or ultraviolet light. These hydrogels are made of macromers including a water-soluble region, a biodegradable region, and at least two polymerizable regions as described in U.S. Pat. No. 5,410,016. For example, the hydrogel can begin with a biodegradable, polymerizable macromer including a core, an extension on each end of the core, and an end cap on each extension. The core is a hydrophilic polymer, the extensions are biodegradable polymers, and the end caps are oligomers capable of cross-linking the macromers upon exposure to visible or ultraviolet light, e.g., long wavelength ultraviolet light. These types of hydrogels can be used with transparent or translucent molds, or with molds that have optic fibers that introduce light into the mold.

Examples of such light solidified hydrogels include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers comprising these hydrogels can be manipulated by standard techniques to modify their physical properties such as rate of degradation, differences in crystallinity, and degree of rigidity.

Tissue Precursor Cells

Various types of isolated cells or tissue precursor cells (e.g., progenitor or stem cells) can be used in methods according to the invention. However, isolated chondrocytes and fibroblasts are preferred.

Cartilage is a specialized type of dense connective tissue consisting of cells embedded in a matrix. There are several kinds of cartilage, and any one of these can be used in the new methods. Hyaline cartilage is a bluish-white, glassy translucent cartilage having a homogeneous matrix containing collagenous fibers that is found in articular cartilage, in costal cartilages, in the septum of the nose, and in the larynx and trachea. Articular cartilage is hyaline cartilage covering the articular surfaces of bones. Costal cartilage connects the true ribs and the sternum. Fibrous cartilage contains collagen fibers. Yellow cartilage is a network of elastic fibers holding cartilage cells which is found primarily in the epiglottis, the external ear, and the auditory tube. By harvesting the appropriate chondrocyte precursor cells, any of these types of cartilage tissue can be grown using the methods of the invention.

Tissue precursor cells can be obtained directly from a mammalian donor, e.g., a patient's own cells, from a culture of cells from a donor, or from established cell culture lines. Preferably the mammal is a mouse, rat, rabbit, guinea pig, hamster, cow, pig, horse, goat, sheep, dog, cat, and most preferably, the mammal is a human. Cells of the same species and preferably of the same immunological profile can be obtained by biopsy, either from the patient or a close relative. Using standard cell culture techniques and conditions, the cells are then grown in culture until confluent and used when needed. The cells are preferably cultured only until a sufficient number of cells have been obtained for a particular application.

If cells are used that may elicit an immune reaction, such as human fibroblast cells from an immunologically distinct donor, then the recipient can be immunosuppressed as needed, for example, using a schedule of steroids and other immunosuppressant drugs such as cyclosporine. However, the use of autologous cells will avoid such an immunologic reaction.

Cells can be obtained directly from a donor, washed, suspended in a selected hydrogel before being injected into a mold. To enhance cell growth, the cells are added or mixed with the hydrogel just prior to injection.

Cells obtained by biopsy are harvested, cultured, and then passaged as necessary to remove contaminating, unwanted cells. The isolation of chondrocytes is described in the examples below. Fibroblasts and other cells can be isolated in a similar fashion.

Cell viability can be assessed using standard techniques including visual observation with a light or scanning electron microscope, histology, or quantitative assessment with radioisotopes. The biological function or metabolism of the cells can be determined using a combination of the above techniques and standard functional assays.

Examples of cells that can be delivered into molds and subsequently grow new tissue in living tissue constructs include epidermal cells; chondrocytes and other cells that form cartilage (“cartilage-forming cells”); dermal cells; fibroblasts; epithelial cells; endothelial cells; ear canal cells; and cells derived from the tympanic membrane.

Preparation of Hydrogel-Cell Compositions

First, a hydrogel of choice is prepared using standard techniques. For example, a biodegradable, thermosensitive polymer at a concentration ranging between 5 and 25% (W/V) is useful for the present invention. If the hydrogel is an alginate, it can be dissolved in an aqueous solution, for example, a 0.1 M potassium phosphate solution, at physiological pH, to a concentration between 0.1 to 4% by weight, e.g., 2%, to form an ionic hydrogel.

Second, isolated tissue precursor cells are suspended in the polymer solution at a concentration mimicking that of the tissue to be generated. The optimal concentration of cells to be delivered into the mold is determined on a case by case basis, and may vary depending on cellular type and the region of the patient's body into which the living tissue implant is inserted. Optimization experiments require modifying only a few parameters, i.e., the cell concentration or the hydrogel concentration, to provide optimal viscosity and cell number to support the growth of new tissue. For chondrocytes, the cell concentration range is from about 10 million cells/ml to about 100 million cells/ml, e.g., from about 25 million cells/ml to about 50 million cells/ml.

Implantation of Constructs

To implant a construct described herein, the perforation in the patient's eardrum is cleared of any dead cells or tissue, and the construct is implanted directly into the perforation using standard techniques.

Over time, e.g., over a period of approximately six weeks, the constructs including cells will become vascularized and the chondrocytes will grow new cartilaginous tissue that takes the shape of the repair construct and engrafts to existing tympanic membrane tissue. In constructs that do not include cells, cells from the surrounding membrane tissue, e.g., fibroblasts followed by keratinocytes, will grow into the space of the perforation as the hydrogel construct degrades, eventually repairing the perforation in the existing tympanic membrane tissue.

Packaging and Storage of Acellular Hydrogel Constructs

In some embodiments, the acellular hydrogel constructs described herein are packaged for storage before use. Such packaging can be either wet or dry. Both wet and dry packaging methods and packages are within the scope of the present invention. In general, all storage methods should maintain the constructs in a sterile condition.

For wet packaging methods, the degradable hydrogel constructs are placed, ideally individually, in a sterile, sealable container filled with a sterile saline solution. In some embodiments, the sterile saline will include CaCl₂, e.g., about 0.05-0.1 M CaCl₂, to maintain the crosslinks in the hydrogel and retain the shape of the construct. Suitable containers include blister packs or individually molded plastic wells that are sealable with foil covers.

For dry packaging methods, the degradable hydrogel constructs can be freeze dried using standard methods, and packaged in a sterile container. Such freeze-dried constructs can be sterilized, e.g., using ethylene oxide, before packaging. In these methods, the dried constructs can be packed in a kit with a container of sterile saline suitable for rehydrating the dried constructs before implantation, and instructions for doing so.

### EXAMPLES

### Example 1

### Construction of Molds and Alginate Construct Formation

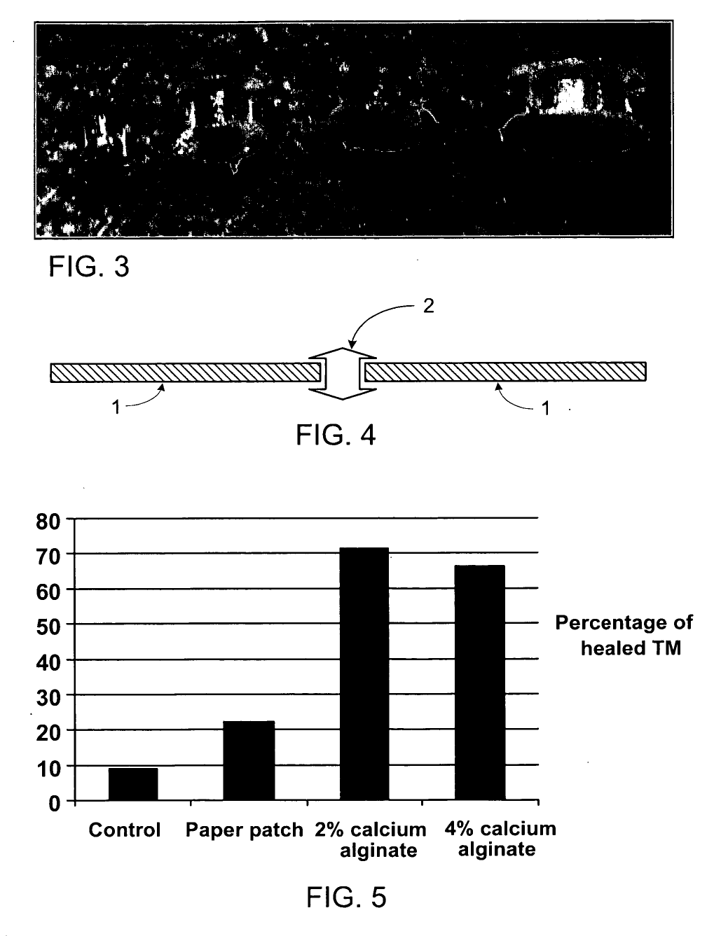

A schematic illustration of a method for making the hydrogel constructs is shown in FIG. 1A. Briefly, a mold for fabricating hydrogel constructs was designed using SolidWorks 2000 (Computer-Aided Products, Peabody, Mass.) and fabricated out of acrylonitrile butadiene styrene (ABS) using fused deposition modeling using the Stratasys Prodigy platform (Stratasys, Inc., Eden Prairie, Minn.). Solutions of 2% and 4% ultrapure low-viscosity alginate (Novamatrix, Princeton, N.J.) in phosphate-buffered saline (PBS) (Invitrogen, Carlsbad, Calif.) were sterilized by filtration through a 0.22 μm filter (Millipore, Billerica, Mass.) and mixed with an autoclaved solution 20 mg/mL CaSO4 in PBS a 2:1. The combination was mixed for 10 seconds using two syringes and a three-way stopcock, then injected into the mold using a sterile 18-gauge needle. Over time, CaSO₄ crosslinks individual alginate molecules resulting in increasing stiffness and resilience of the resultant compound and enabling the generation of complex geometries that retain their shape in vitro (Hott et al., Laryngoscope 114:1290-1295 (2004)) and in vivo (Chang et al., J. Biomed. Mater. Res. 55:503-511 (2001)). In contrast to the technique originally described by Hott et al., chondrocytes were not used as part of the scaffold. The alginate constructs were compounded with both 2% and 4% calcium solution to compare the relative mechanical properties and identify whether the concentration of calcium has an impact on healing potential. As used, the calcium alginate constructs were self-retaining and required no modification. The structure and implantation technique of the constructs is similar to a standard biflanged tympanostomy tube, as illustrated in FIG. 2. Hydrogel constructs were prepared in different sizes as measured by the diameter of the interflange shaft. Sizes included 2-mm, 3-mm, 4-mm, and 5-mm constructs (FIG. 3).

### Example 2

### Implantation of Hydrogel Constructs in an Animal Model of Tympanic Membrane (TM) Perforation

To evaluate the efficacy of the hydrogel constructs in vivo, a chinchilla animal model was used, as the chinchilla has been identified as an effective model that simulates TM perforations in humans (Amoils et al., Otolaryngol. Head Neck Surg. 106:47-55 (1992)).

Experimental Methods

Sixty ears from 30 adult female chinchillas were used. The procedure was performed under general anesthesia by maintaining the animals under spontaneous mask ventilation using isoflurane. An approximately 5-mm central perforation was created using the thermal cautery device (Gyrus ENT, Memphis, Tenn.). At 3 weeks, ears were reinspected and further cautery was applied if any degree of closure had occurred to maintain a 5-mm central perforation. All procedures were performed through a standard ear speculum under direct microscopic visualization as described by Amoils et al., (1992) supra.

All animals underwent bilateral tympanic membrane perforation using the cautery device as previously described (Amoils et al., (1992) supra). A total of 60 ears were included in the study. At 6 weeks, ears with stable TM perforation were randomized to one of four groups: treatment with 2% calcium alginate constructs, treatment with 4% calcium alginate constructs, treatment with standard paper patch, and an untreated control group. At 2, 4, and 6 weeks, representative specimens were obtained from the experimental group for inspection and histologic analysis to evaluate the healing process. At approximately 10 weeks postimplantation, all remaining animals were killed with direct inspection and harvest of the TMs for histologic analysis and determination of healing.

Hearing sensitivities were measured using auditory brainstem response (ABR) thresholds. All audiologic evaluation was performed using an Intelligent Hearing Systems (IHS) ABR system version 3.6x (Intelligent Hearing Systems, Miami, Fla.). A custom-made sound source calibrated by the manufacturer was used to fit securely to the external auditory canal of the chinchilla. The animal was placed in a soundproof box and a click stimulus was delivered directly to the conchal bowl. Anesthesia was performed using intraperitoneal injection of rodent cocktail (xylazine/ketamine) owing to the need to isolate the animals in the sound proof field.

Baseline ABR testing was performed on two animals before any manipulation of the TM. Three animals were tested after creation of bilateral TM perforations using the thermal cautery. Each of the three animals underwent implantation of a calcium alginate construct in the right ear. At six weeks postimplantation, those three animals underwent bilateral ABR testing and comparison was made to the control animals and between the implanted and nonimplanted ears. The purpose of measuring hearing thresholds is by no means an assessment of functional hearing outcome after treatment, but an objective measure to determine the presence of potential ototoxicity resultant from the hydrogel compound.

Results

Of the 60 ears included in the study, 27 were excluded as a result of uncontrolled infection (after surgery for creation of the perforation but before construct placement) or illness and death affecting the animal. The remaining 43 ears were randomized to four groups: 11 to treatment with 4% calcium alginate constructs, 11 to treatment with 2% calcium alginate constructs, 10 to treatment with traditional paper patch, and 11 were assigned to a no treatment or control arm. The control arm was included to evaluate the long-term stability of the perforation and the natural history of the healing process.

Of the experimental arm treated with calcium alginate constructs and paper patch, a total of five animals (10 ears) were killed at 2, 4, and 6 weeks postimplantation to objectively establish a chronologic analysis of the healing process. All remaining animals were killed at approximately 10 weeks postimplantation. Their TM were directly inspected and subjected to histologic analysis. At 10 weeks, final evaluation revealed complete resolution of the perforation in four of six ears in the arm treated with 4% calcium alginate, five of seven in the arm treated with 2% calcium alginate, two of nine in the arm treated with paper patch, and in one of 11 in the control or untreated group (FIG. 5).

Histologic analysis demonstrated the presence of portions of the alginate constructs up to 10 weeks after implantation. Areas surrounding the residual alginate were composed of layers of fibroblast-populated connective tissue surrounded by epithelium. There was no evidence of foreign body-type giant cells, granulomatous inflammation, or other cellular indicators of chronic foreign body response to the implants.

The χ² statistical test was used to analyze the statistical significance of comparative results. P values were obtained between the experimental and control groups and between the calcium alginate construct and paper patch group. The paper patch group demonstrated no statistically significant advantage, over the control group (χ²=0.668, P>0.1), whereas both the 2% and 4% calcium alginate groups demonstrated significant advantage over the control group (χ²=6.2, P<0.02 and χ²=7.48, P<0.01, respectively).

There was a statistically significant difference noted between the 2% calcium alginate and the paper patch-treated group (χ²=3.86, P<0.05), but no difference was found between the 4% calcium alginate and the paper patch group (χ²=2.96, P>0.05). ABR testing of native chinchillas demonstrates moderate test-to-test variability based on subtle differences in probe position within the conchal bowl. The two control animals showed an absolute range of hearing from 25 to 40 dB with ear-to-ear variability averaging 10 dB. The three animals tested after heat cautery perforation and repair of the right-sided TM perforation with an alginate-based construct demonstrated similar findings with a threshold range from 30 to 45 dB and ear-to-ear variability ranging between 5 and 10 dB. Of the three experimental animals, two had higher thresholds in the repaired ear (5 and 10 dB greater), and one had a higher threshold in the non-repaired ear (15 dB). These preliminary results support the absence of significant ototoxicity resulting from application of the construct to the affected ear.

Discussion

The results described herein demonstrate that hydrogel constructs such as calcium alginate-based constructs provide a safe, rapid, and effective mean to repair small- to moderate-sized chronic tympanic membrane™ perforations. In an animal model, calcium alginate compounds appear to offer a definite advantage in promoting the healing process when compared with traditional paper patching. The absorbable alginate scaffold promotes cellular growth and matrix formation. The validity of the experimental model and findings is further reinforced by the excellent long-term patency of our control perforations. Therefore, calcium alginate constructs offer a distinct advantage over the current techniques for repair of small TM perforations owing to their ease of use and greater efficacy.

There was a little difference in the efficacy of the 2% versus the 4% calcium alginate constructs that were evaluated. The 4% calcium alginate compound was noticeably more rigid and resistant to handling trauma. Through the experimental process, a clear preference for 4% calcium alginate construct secondary to superior ease of handling was developed. The untreated arm offered a good internal control as the natural history of TM perforation is characterized by a high rate of spontaneous healing. No significant difference was noted between the untreated ears and those repaired with paper patch. The paper patch technique, often used in the clinical setting, suffers from limited efficacy. The success rate is greatly diminished as the size of the perforation increases (Nichols et al., Arch. Otolaryngol. Head Neck Surg. 124:417-419 (1998)). Compared with paper patch, calcium alginate constructs offer a similar ease and safety; however, the efficacy and success rate are significantly greater. This may mitigate the need for a more formal tympanoplasty in the repair of failed paper patch myringoplasties in the clinical setting. Compared with the inlay cartilage graft technique (Eavey's technique), the biflanged constructs described herein have the potential to eliminate the need to harvest an autologous graft and hence decrease morbidity and operative time.

### Example 3

### Isolation of Chondrocytes

Freshly slaughtered calf forelimbs were obtained from a local slaughterhouse within 6 hours of sacrifice. The forelimbs were dissected under sterile conditions to expose the is articular surfaces of the glenohumeral and humeroulnar joint. Cartilage fragments were sharply curetted off the articular surface of each joint, were subjected to collagenase II digestion (3 mg/ml) (Worthington Biochemical Corp, freehold, N.J. USA.) at 37° C. for 12 to 18 hours. Preparation of chondrocytes was in accordance with methods described in Klagsburn, 1979, Meth. Enzymol., 58:560-564.

The resulting cell suspension was passed through a sterile 25μ polypropylene mesh filter (Spectra/Mesh 146-426 Spectrum Medical Industries, Inc., Laguna Hills, Calif., and USA.). The filtrate was centrifuged at 6000 rpm, and the resulting cell pellet was washed twice with copious amounts of Dulbecco phosphate buffered-saline (PBS) (Gibco, Grand Island, N.Y., USA) without Ca²⁺. Cell number was determined using a hemocytometer and the cell viability determined using trypan blue dye (Sigma-Aldrich, Irvine, KA, USA.). Chondrocyte suspensions were concentrated to various cellular densities of 10, 25, and 50×10⁶ cells/ml and suspended in a solution of 2% alginate.

### Example 4

### Construction of Molds

A three-dimensional reconstruction of a positive template for a tissue-engineered patch for a tympanic perforation was generated by computer-aided design (CAD) using standard techniques. FIG. 2 illustrates an exemplary virtual template. This image is ported directly to software in a mold-making device, which generates a negative mold.

### Example 5

### Alginate Construct Formation

Isolated fibroblast and cartilage cells are resuspended in a 2% sterile sodium alginate (Pronova Biopolymer, Norway) solution (0.1M K₂HPO₄, 0.135M NaCl, pH 7.4), which has previously been sterilized with a 0.45 nm filter to yield various cellular concentrations of 10, 25, and 50×10⁶/ml alginate solution. Immediately before injection into the silicon mold, sterilized CaSO₄ (0.2 gm/ml of alginate) in PBS solution is mixed with chondrocyte-alginate construct to initiate gel formation. The chondrocyte-fibroblast/alginate/CaSO₄ mixture is delivered into the sterilized mold of Example 2 using a 10 ml syringe and an 18.5 gauge needle. Formed shapes are removed from molds 10 minutes after injection. FIG. 1B illustrates the overall method.

The solidified construct can be put into culture under standard conditions, e.g., for one week, to allow the cells to grow to confluence within the hydrogel construct. Alternatively, the construct can be implanted directly into a patient.

### OTHER EMBODIMENTS

A number of embodiments of the invention have been described. Nevertheless, it will be understood that various modifications may be made without departing from the spirit and scope of the invention.

What is claimed is:

1. A unitary degradable hydrogel construct having a first, middle portion having a first end and a second end, wherein the first end is coaxially connected to a second portion having a larger diameter than the first, middle portion, and the second end is coaxially connected to a third portion having a larger diameter than the first, middle portion.

2. The unitary degradable hydrogel construct of claim 1, wherein the terminal ends of the second and third portions that are not connected to the first, middle portion are dome-shaped.

3. The unitary degradable hydrogel construct of claim 1, wherein the middle portion has a circular cross-sectional shape.

4. The unitary degradable hydrogel construct of claim 1, wherein the construct is freeze-dried.

5. The unitary degradable hydrogel construct of claim 1, wherein the hydrogel construct comprises tissue precursor cells.

6. The unitary degradable hydrogel construct of claim 5, wherein the tissue precursor cells are chondrocytes or fibroblasts, or a combination thereof.

7. The unitary degradable hydrogel construct of claim 5, wherein the tissue precursor cells are chondrocytes.

8. The unitary degradable hydrogel construct of claim 1, wherein the construct does not include tissue precursor cells.

9. The unitary degradable hydrogel construct of claim 1, wherein the hydrogel is selected from the group consisting of polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

10. The unitary degradable hydrogel construct of claim 1, wherein the hydrogel is selected from the group consisting of alginate, chitosan, pluronic, collagen, and agarose.

11. A kit comprising the unitary degradable hydrogel construct of claim 1, suspended in a sterile saline solution, and instructions for use in repairing a perforation in a tympanic membrane in a mammal.

12. A kit comprising the unitary degradable hydrogel construct of claim 1, wherein the construct is freeze-dried, and instructions for use in repairing a perforation in a tympanic membrane in a mammal.

13. The kit of claim 12, further comprising a container of sterile saline suitable for rehydrating the freeze-dried construct.

14. A method of making a hydrogel construct for repairing a perforation in a tympanic membrane, the method comprising:
providing a negative mold having a negative shape of a construct having a first, middle portion having a first end and a second end, wherein the first end is coaxially connected to a second portion having a larger diameter than the first, middle portion, and the second end is coaxially connected to a third portion having a larger diameter than the first, middle portion;
introducing a liquid hydrogel composition into the mold;
inducing gel formation to solidify the liquid hydrogel composition to form the construct; and
removing the hydrogel construct from the mold after gel formation.

15. The method of claim 14, wherein the hydrogel is selected from the group consisting of alginate, chitosan, pluronic, collagen, and agarose.

16. The method of claim 14, wherein the hydrogel is alginate.

17. The method of claim 16, wherein the alginate concentration is from 0.5% to 8%.

18. The method of claim 16, wherein the alginate concentration is from 1% to 4%.

19. The method of claim 16, wherein the alginate concentration is approximately 4%.

20. The method of claim 14, wherein inducing gel formation comprises contacting the liquid hydrogel with a suitable concentration of a divalent cation.

21. The method of claim 20, wherein the divalent cation is Ca⁺⁺.

22. The method of claim 21, wherein the suitable Ca⁺⁺ concentration is 0.2 g/ml of the liquid hydrogel composition.

23. The method of claim 14, further comprising packaging the degradable hydrogel construct for long term storage.

24. The method of claim 14, wherein the negative mold is prepared using CAD/CAM or rapid prototyping.

25. A method of repairing a perforation in a tympanic membrane in a mammal, the method comprising:
providing a suitable negative mold having a negative shape of a construct having a first, middle portion having a first end and a second end, wherein the first end is coaxially connected to a second portion having a larger diameter than the first, middle portion, and the second end is coaxially connected to a third portion having a larger diameter than the first, middle portion;
introducing a liquid hydrogel composition into the mold;
inducing gel formation to solidify the liquid hydrogel composition to form the construct;
removing the construct from the mold after gel formation; and
implanting the construct into the perforation in the tympanic membrane in the mammal.

26. A method of repairing a perforation in a tympanic membrane in a mammal, the method comprising obtaining implanting the construct of claim 1 into the perforation in the tympanic membrane in the mammal.
